(19) United States
(12) Patent Application Publication
(10) Pub. No.: US 2021/0345877 A1
(43) Pub. Date: Nov. 11, 2021

(54) METHOD AND DEVICE FOR SELF-MEASUREMENT OF INTRA-OCULAR PRESSURE

(71) Applicants: NATIONAL UNIVERSITY HOSPITAL (SINGAPORE) PTE LTD, Singapore (SG); NATIONAL UNIVERSITY OF SINGAPORE, Singapore (SG)

(72) Inventors: Victor Teck Chang KOH, Singapore (SG); Clement Woon Teck TAN, Singapore (SG); Wing-Kin TAM, Hong Kong (CN); Zhi YANG, Edina, MN (US); Baitong WANG, Guangzhou (CN)

(21) Appl. No.: 17/268,600

(22) PCT Filed: Aug. 16, 2019
(86) PCT No.: PCT/SG2019/050406

§ 371 Date: Feb. 16, 2021

(30) Foreign Application Priority Data
Aug. 16, 2018 (SG) 10201806935Y

(51) Int. Cl. A61B 3/16; A61B 5/00; G06N 3/08; G16H 50/20

(52) U.S. Cl. CPC A61B 3/16; A61B 5/7267; A61B 2562/04; G16H 50/20; A61B 2562/0247; G06N 3/08

(57) ABSTRACT

A self-tonometry device for measuring intra-ocular pressure in an eye of a subject, may include a plurality of sensors and a processor for executing a machine learning module. The plurality of sensors may be arranged in an array for measuring a plurality of pressures at respective positions on an eye of a subject, when the plurality of sensors in the array apply a force to the eye at the respective positions through an eyelid of the subject. The processor may be configured to receive the plurality of pressures at the respective location from the plurality of sensors, and to compute using the machine learning module, an intra-ocular pressure in the eye based on the plurality of pressures measured at the respective positions through the eyelid of the subject.

### TECHNICAL FIELD

The present disclosure generally relates to intra-ocular pressure measurements, and more specifically to a self-tonometry device for monitoring intra-ocular pressure.

### BACKGROUND

Glaucoma is the second most common cause of blindness in the world. It may be characterized by irreversible optic nerve damage. A raised intraocular pressure (IOP) remains the only modifiable risk factor. The majority of treatment strategies for glaucoma are aimed at reducing IOP. Thus, IOP monitoring is the single most important measurement for detecting and assessing glaucoma in the eyes of a patient.

The current gold standard for measuring IOP is Goldmann Applanation Tonometry (GAT), which is a slit-lamp mounted IOP measurement device typically requiring topical anaesthesia and performed by a trained ophthalmologist. Despite these limitations, probably the single most important shortcoming is that the GAT IOP measurement represents a single 2-3 second “snapshot” of a patient's IOP. However, there may be a normal diurnal variation of IOP, which may fluctuate even more so in eyes with glaucoma. These fluctuations may have significant inter-individual variation depending on age, activities and medical co-morbidities.

Due to these diurnal IOP variations, a measurement of the maximum IOP may easily be missed when the patient is not in the clinic. Furthermore, studies have shown that the amplitude of IOP fluctuations and maximum IOP may be associated with glaucoma progression. This may explain why the vision of one-third of glaucoma patients may continue to worsen despite seemingly adequate IOP control and reduction.

Thus, there may be a need for a self-administered, highly reproducible IOP-measuring device that permits 24-hour monitoring of IOP.

### SUMMARY

There is thus provided, in accordance with some embodiments of the present disclosure, a self-tonometry device for measuring intra-ocular pressure in an eye of a subject. The self-tonometry device may include a plurality of sensors and a processor for executing a machine learning module. The plurality of sensors may be arranged in an array for measuring a plurality of pressures at respective positions on an eye of a subject, when the plurality of sensors in the array apply a force to the eye through an eyelid of the subject. The processor may be configured to receive the plurality of pressures at the respective positions measured from the plurality of sensors as an input to the machine learning module, and to compute using the machine learning module, an intra-ocular pressure in the eye based on the plurality of pressures at the respective positions measured through the eyelid of the subject.

### BRIEF DESCRIPTION OF THE DRAWINGS

Some non-limiting embodiments or features of the disclosed subject matter are illustrated in the following drawings.

In the drawings:

FIG. 1A schematically illustrates a side view of an eye and a self-tonometry device before sensors contact an eyelid, in accordance with some embodiments of the present disclosure:

FIG. 1B schematically illustrates a side view of an eye and self-tonometry device while sensors contact an eyelid, in accordance with some embodiments of the present disclosure;

FIG. 1C schematically illustrates a front view of an eye with exemplary positions of sensors on an eyelid, in accordance with some embodiments of the present disclosure;

FIG. 2 schematically illustrates a side view of a self-tonometry device, in accordance with some embodiments of the present disclosure;

FIG. 3 schematically illustrates a block diagram of a self-tonometry system, in accordance with some embodiments of the present disclosure;

FIG. 4 schematically illustrates an artificial neural network (ANN) model for eye pressure computations, in accordance with some embodiments of the present disclosure;

FIG. 5 schematically illustrates an experimental setup for testing self-tonometry device on a mechanical model, in accordance with some embodiments of the present disclosure:

FIG. 6A illustrates graphs of sensor pressure measurements versus time for different intra-luminal pressures on the mechanical model, according to some embodiments of the present disclosure;

FIG. 6B illustrates a graph of absolute error versus data sample, in accordance with some embodiments of the present disclosure;

FIG. 6C illustrates a graph of a real pressure and a predicted pressure versus data sample, in accordance with some embodiments of the present disclosure;

FIG. 6D illustrates a graph of sample count versus absolute error, in accordance with some embodiments of the present disclosure;

FIG. 7 schematically illustrates an experimental setup for testing a self-tonometry device on a pig eye, in accordance with some embodiments of the present disclosure;

FIG. 8A illustrates a graph of absolute error versus data sample, in accordance with some embodiments of the present disclosure;

FIG. 8B illustrates a graph of the real pressure and the predicted pressure versus data sample, in accordance with some embodiments of the present disclosure;

FIG. 8C illustrates a graph of sample count versus absolute error, in accordance with some embodiments of the present disclosure;

FIG. 9 illustrates graphs of sensor pressure measurements versus time using a self-tonometry device on an eye, according to some embodiments of the present disclosure;

FIG. 10 illustrates a graph of sensor pressure measurements comparing predicted pressure with true actual tested pressure using a self-tonometry device on an eye 10, according to some embodiments of the present disclosure; and

FIG. 11 illustrates a graph of normalized sensor pressure measurements comparing predicted pressure with true actual tested pressure using a self-tonometry device on an eye, according to some embodiments of the present disclosure.

### DETAILED DESCRIPTION

Some embodiments of the present disclosure provide a self-tonometry device for measurement of intra-ocular pressure (IOP) in the eye of a subject. The self-tonometry device may use a plurality of pressure sensor measurements measured by a respective plurality of sensors arranged in an array that may be pressed onto the outside surface of an eyelid of a subject. By pressing the sensor array onto the outside surface of the eyelid, a force is applied through the eyelid onto an eye of the subject. The plurality of pressure sensors may measure a respective plurality of pressures at the respective positions along the eyeball at the inner surface of the eyelid substantially opposite to the sensor array on the outer surface of the eyelid. The self-tonometry device may compute the IOP based on the plurality of pressure sensor measurements measured through the eyelid.

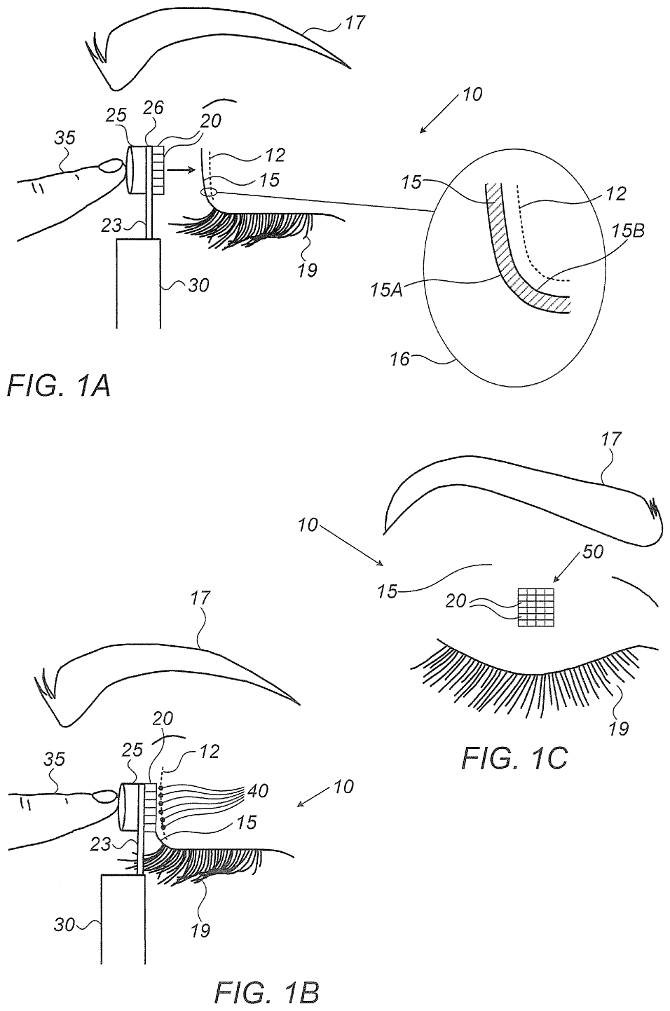

FIG. 1A schematically illustrates a side view of an eye 10 and a self-tonometry device 30 before sensors 20 contact an eyelid 15, in accordance with some embodiments of the present disclosure. Self-tonometry device 30 may include a plurality of sensors 20 arranged in an array and coupled to a flexible member 23 at an end 26 of self-tonometry device 30. Eye 10 may include an eyebrow 17 and eyelashes 19 on eyelid 15.

A finger 35 may be used to press the plurality of sensors 20 into eyelid 15 using a force-transfer assembly 25 so as to allow sensors 20 to contact eyelid 15 to perform pressure measurements on an eyeball 12 of eye 10 through eyelid 15 typically when the eyelids of the subject are closed. As shown in an enlargement 16, pressure sensors 20 may contact an outer surface 15A of eyelid 15 to perform pressure measurements on eyeball 12 opposite an inner surface 15B of eyelid 15. The gap shown in enlargement 16 between inner surface 15B and eyeball 12 is shown merely for visual clarity, and it should be clear to one skilled in the art that eyeball 12 is typically in contact with inner surface 15B.

FIG. 1B schematically illustrates a side view of eye 10 and self-tonometry device 30 while sensors 20 contact eyelid 15, in accordance with some embodiments of the present disclosure. When finger 35 of a subject presses on force-transfer assembly 25 to perform self-tonometry measurements, flexible member 23 permits the plurality of sensors 20 to be pushed into contact with eyelid 15 at outer surface 15A. The applied force by finger 35 may enable sensors 20 to measure the pressure at positions 40 on eyeball 12 through eyelid 15. According to some embodiments, sensors 20 may be six sensors, though other numbers of sensors may be implemented. Each of the six sensors 20 illustrated in FIG. 1B are substantially opposite to each of six respective positions 40 on eyeball 12 at which eye pressure is measured by the sensors in this exemplary embodiment.

FIG. 1C schematically illustrates a front view of eye 10 with exemplary positions of sensors 20 on eyelid 15, in accordance with some embodiments of the present disclosure. The side view of eye 10 shown in FIGS. 1A and 1B show a planar cut through six sensors 20 arranged linearly. However, in some embodiments of the present disclosure, self-tonometry device 30 may include a miniaturized sensor array 50 with typically 12-40 sensors 20, for example, with 12-24 respective output channels to measure the eye pressure over eyelid 15 in a consistent manner. FIG. 1C illustrates an exemplary 6 row by 4 column array of 24 sensors placed on eyelid 15. Accordingly, there are 24 pressure measurements made by the 24 sensors at 24 respective positions 40 on eyeball 15. Sensor array 50 with 6 rows and 4 columns is shown in FIG. 1C merely for conceptual clarity and not by way of limitation of the embodiments of the present disclosure. Any number of sensors 20 arranged in any suitable array 50 of rows and columns may be used.

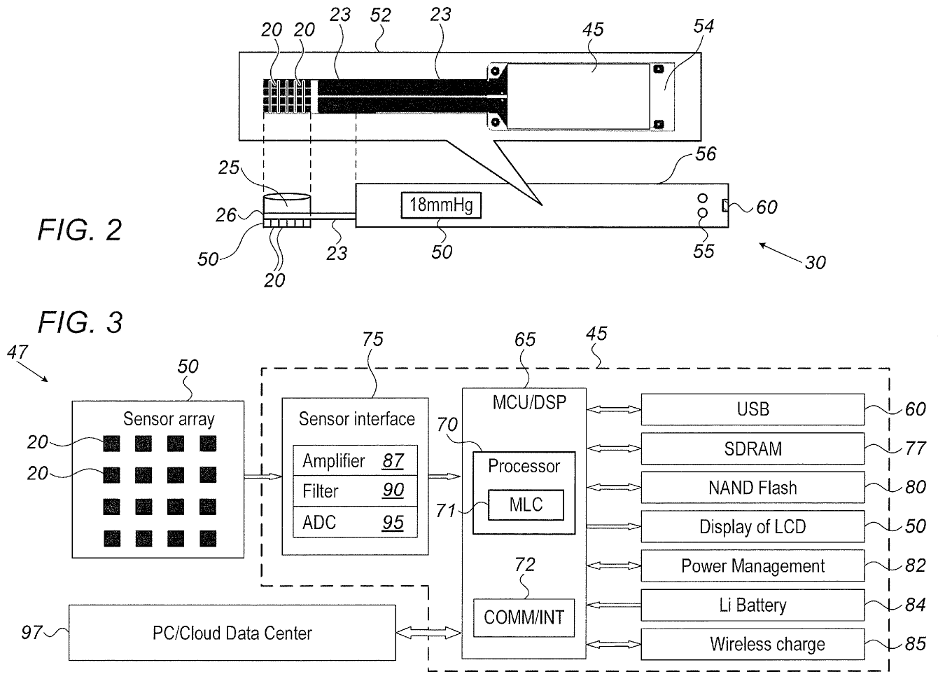

FIG. 2 schematically illustrates a side view of self-tonometry device 30, in accordance with some embodiments of the present disclosure. Self-tonometry device 30 may include a housing 56 which may have a size and/or form factor of a standard marker pen, for example. The subject, such as a patient, may easily perform IOP self-measurements on their own eyes using self-tonometry device 30 according to the patient's need to periodically assess if the subject measured high IOP measurements anytime during the day.

In some embodiments of the present disclosure, housing 56 may include a display 50 for displaying the intra-ocular pressure (IOP) or other metrics, and/or electrical buttons 55 such as an on/off power and/or calibration button. As shown in an enlargement 52, a substrate 54, such a flexible circuit board, may be housed within housing 56. Substrate 54 may include flexible member 23 and circuitry 45. Circuitry 45 may be electrically coupled to each of sensors 20 in sensor array 50 at end 26 via electrical conductors formed and/or bonded and/or placed on flexible member 23. In some embodiments, flexible member 23 may be formed from a same piece as substrate 54, which may include flexible member 23 and circuitry 45. In other embodiments, flexible member 23 may be formed from a separate piece of substrate 54 which may be used for circuitry 45, and bonded or connected to flexible member 23 separate from substrate 54.

In some embodiments of the present disclosure, sensor array 50 may include of an array of capacitive pressure sensors. Capacitive sensors are typically more sensitive than normal resistive pressure sensors, and thus, may be able to detect the minor changes of IOP due to pathological conditions in eye 10. Array 50 of sensors 20 are used in self-tonometry device 30 instead of a single sensor, so as to provide multiple readings of the eye pressure from different positions 40, which further improves the accuracy of the IOP measurement.

In some embodiments of the present disclosure, sensor array 50 may be integrated with 4×6 capacitive pressure elements, or sensors 20. The number of sensors typically range, for example from 12-40 sensors. The sensing area or array 50 may have a total area of 8×12 mm. The sensing area may have a typical range of 8-14 mm×12-18 mm, for example. The size of each sensor may typically be 2×2 mm. The thickness of the sensors array mounted on flexible member 23 at end 26 may be 0.5 mm. Due to the small thickness, end 26 may be flexible to press sensors 20 on eyelid 15 using finger 35 for performing the IOP measurement.

In some embodiments of the present disclosure, typical sensor array 50 specifications may include, for example:

Thickness: 0.5 mm

Force Ranges: 0-2 psi.

Linearity (Error): <+3%

Repeatability: <+2.5% of full scale

Hysteresis: <4.5% of full scale

Drift: <5% per logarithmic time scale

Response Time: <5 μsec

FIG. 3 schematically illustrates a block diagram of a self-tonometry system 47, in accordance with some embodiments of the present disclosure. System 47 may include sensor array 50 with the plurality of sensors 20 coupled to circuitry 45. Circuitry 45 may be configured to relay data measured and computed by circuitry 45 in self-tonometry device 30 to a PC/Cloud Data Center 97.

Circuitry 45 may include a sensor interface 75 coupled to a microcontroller/digital signal processing (MCU/DSP)unit 65. MCU/DSP 65 may be coupled to a USB(input/output data interface) 60, a memory 77, such as SDRAM, NAND flash memory 80, LCD display 50, power management circuitry 82, a Li battery 84 for powering self-tonometry device 30, and a wireless charging module 85. Sensor interface 75 may include an amplifier 87, a filter 90, and an analog-to-digital converter 95. MCU/DSP 65 may include a processor 70 for performing the signal processing in the pressure measurements and executing a machine learning module for analyzing the pressure measurements for computing the IOP in eye 10. MCU/DSP may include a communication module and interface 72 with circuitry for relaying data between self-tonometry device 30 and any other computing and/or storage device (e.g. PC/Cloud Data Center 97) via any suitable wired and/or wireless communication protocols (e.g., Wi-Fi, Bluetooth, cellular communication).

In some embodiments of the present disclosure, signals from sensors 20 in array 50 may be relayed to sensor interface 75. Sensor interface 75 may amplify, filter, and digitize the signal using ADC 95. The digitized pressure measurement signals may then be relayed MCU/DSP 65 for signal analysis. The IOP analysis results may be displayed on integrated LCD screen 50 for the subject to observe. SD-card in a slot may be installed in self-tonometry device 30 for recording historical IOP data for later reference. Power management circuitry 82 may be used to switch off self-tonometry device 30 automatically when not in use to save power. In some embodiments of the present disclosure, all of the components shown in FIG. 3 may be miniaturized to fit within the size of a normal marker pen, making self-tonometry device 30 portable and easy to use.

Processor 70 (e.g., MCU/DSP unit 65) may include one or more processing units. e.g. of one or more computers. Processor 70 may be configured to operate in accordance with programmed instructions stored in SDRAM 77 and/or NAND Flash 80 or any other suitable storage device and/or memory. In operation, processor 70 may execute a method for measuring intra-ocular pressure in an eye of a subject.

In some embodiments of the present disclosure, the plurality of pressure measurements at the respective positions may be relayed to processor 70 executing a machine learning module (MLM) 71 in self-tonometry device 30 employing machine learning regression or classification, as an input to a trained artificial neural network (ANN) model. The processor may use machine learning regression or classification through MLM 71 to calculate the IOP based on the plurality of pressure measurements at the respective positions 40.

In the context of the present disclosure, the term “machine learning module” refers to an algorithm, model, function, or code that implements regression or classification. Regression is a predictive machine learning approach in which a computer program may approximate from the data input to a computing device a continues output variable. Classification is a predictive machine learning approach in which a computer program may approximate from the data input to a computing device a discrete output variable. Machine learning model or function may include, for example, an artificial neural network (ANN) model or a random forest model. Although many of the embodiments herein are with reference to the ANN model for computing IOP from the plurality of pressure measurements, self-tonometry device 30 may perform similar calculations using any suitable MLM 71, such as random forest models. Self-tonometry device 30 is not limited to calculating IOP using the ANN model.

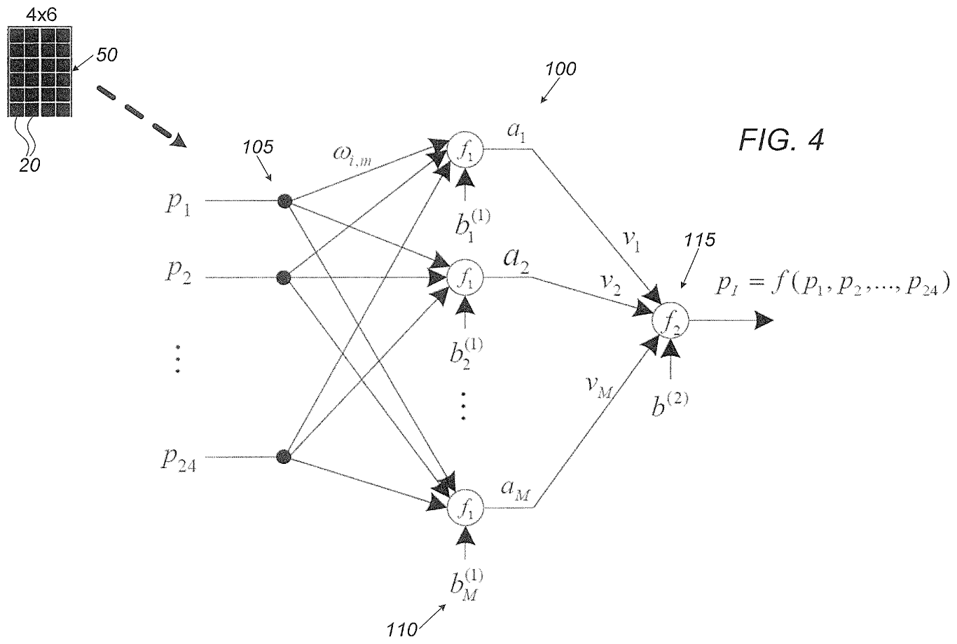

FIG. 4 schematically illustrates an artificial neural network (ANN) model 100 for eye pressure computations, in accordance with some embodiments of the present disclosure. ANN 100 uses a 4×6 sensor array 50, or 24 sensors (e.g., sensors 20), which is merely for conceptual clarity and not by way of limitation of the embodiments of the present disclosure. Any number of sensors may be used in any suitable array configuration.

MLM 71 such as artificial neural network (ANN) 100 may be trained to compute the intra-ocular pressure (TOP). In general, ANN 100 may mimic the architecture of the brain, for example, by passing values from one layers of artificial neurons to another. In the case of ANN 100 shown in FIG. 4, ANN 100 may include an input layer 105, a hidden layer 110, and an output layer 115. ANN 100 may be used to approximate any measurable functions.

For self-tonometry device 30, if the surface pressure on eye 10 (e.g., on eyeball 15 of the subject) has any consistent relationship with the intraocular pressure, the trained ANN may use this relationship to compute the IOP. Furthermore, the use of multiple miniaturized pressure sensors 20 in sensor array 50 may permit multiple pressure samples at predefined positions 40 on the eye (e.g., eyeball 15) for use in the IOP computation to improve the measurement accuracy. For example, noise and pressure variations in measurements from one sensor may be cancelled by measurements from the other sensors in the sensor array, assuming the noise is random.

In some embodiments of the present disclosure, ANN 100 may assume that the intra-ocular (IOP) pressure denoted p_{I} may be a nonlinear function of the applied pressures (p1, p2, . . . , p24) (e.g., by finger 35) from sensor array 50 as given by Eqn. (1):

p_{I}=f(p₁,p₂, . . . ,p₂₄) (1)

An artificial neural network (ANN), such as ANN 100 shown in FIG. 4, has many features, such as the ability to approximate arbitrary functions, the simplicity of learning from samples, and a robustness against noise utilized here to model the function shown in Eqn. (1). ANNs may be used to model nonlinear systems.

As shown in FIG. 4, p₁, p₂, . . . , p₂₄ are the pressures from sensors 20 measured at positions 40, respectively, and are presented as the input to ANN 100 at input layer 105. p_{I}=f(p₁, p₂, . . . , p₂₄) is the intra-ocular pressure at output layer 115, which is a nonlinear function of the pressures (p1, p2, . . . , p24) from sensors 20. f₁ is a sigmoid activation function in hidden layer 110. f₂ is a linear output function in outer layer 115. The output p_{I}=f(x) may be given by Eqn. (2):

$${p}_{I} = {v}^{T} {a}^{( 1 )} + {b}^{( 2 )} = {v}^{T} \frac{1}{1 + exp ( - {ω}^{T} X + {b}^{( 1 )} ) )} + {b}^{( 2 )} = {\sum }_{m = 1}^{M} ( \frac{{v}_{m}}{1 + exp ( - ( {\sum }_{i = 1}^{24} {ω}_{mi} {p}_{i} + {b}_{m}^{( 1 )} ) )} + {b}^{( 2 )} ) \qquad ( 2 )$$

where M is the number of the hidden neurons which may be optimized using experimental data. For example, M=5 after preliminary testing. ω=(ω_{1,1}, ω_{1,2}, . . . , ω_{24,M})^{T} is the matrix of the weights connecting the nodes in input layer 105 with neurons of hidden layer 110. b⁽¹⁾=(b₁⁽¹⁾, b₂⁽¹⁾, . . . , b_{M}⁽¹⁾)^{T} is the bias vector of the neurons of hidden layer 110. V=(v₁, v₂, . . . , v_{M})^{T} is the vector of the weights connecting the neurons of hidden layer 110 with the those in output layer 115. b⁽²⁾ is the bias of the neuron of output layer.

In some embodiments of the present disclosure, ANN 100 may include different configurations of multiple neural network layers, including but not limited to fully connected layer, convolution layer, normalized layer and drop out layer, briefly explained below. The machine learning may be performed via gradient descent. In gradient descent, initially the difference between the estimated pressure and the real pressure are computed to find out the loss. Then the gradient of the loss with respect to each weight are subtracted from the weights respectively.

In some embodiments of the present disclosure, the input data to ANN 100 may include the applied force (e.g., from finger 35, for example) and pressure data from sensors 20 in array 50. The input data may be relayed to ANN 100 to undergo multiple linear and non-linear transformation to produce the output. The output is the estimated intra-ocular pressure (IOP).

In some embodiment of the present disclosure, the calibration of ANN 100 may be performed by measuring the pressure on top 15A of eyelid 15 using sensor array 50. The true IOP may be measured using a reference device, such as a Goldmann applanation tonometer, or a water pressure sensor inserted into an animal eye. The plurality of pressures measure by the respective plurality of sensors 20 in array 50 and the true IOP may be input into ANN 100, so as to train and to fine tune the weights in the ANN model during calibration using gradient descent. Calibrating ANN 100 for each patient produces the best IOP measurement accuracy.

ANN 100 may capture the probability distribution of the input data automatically. Transform functions used in ANN 100 may include, but are not limited to the fully connected layer, convolution layer, batch normalization layer and dropout layer. A non-linear mapping such as the rectified linear unit (ReLU) is occasionally applied to the output of a neural network layer, where ReLU may be given by:

f(x)=0 for x<0 (3)

f(x)=x for x≥0 (4)

In some embodiments of the present disclosure, using ANN 100, the IOP may be estimated by the following steps:
1. The input may be feed into ANN 100.
2. The input may be transformed by one or multiple convolutional layers to produce an output.
3. The output may be further transformed by a normalization layer.
4. The output may be further transformed by one or multiple convolution layers.
5. The output may be further transformed by a normalization layer.
6. The output may be further transformed by a dropout layer.
7. The output may be further transformed by one or multiple fully connected layer
8. The IOP value may be estimated.

The detailed configurations and orders of Step 2-6 are variable and may be changed, so as to improve the estimation accuracy.

In a fully connected layer, the input variables are fully connected to the output. In a convolution layer, the input may be multiplied with a smaller matrix (e.g., a kernel) to produce the output. In a normalization layer, the input values may be normalized to have mean zero and a variance of 1 in each feature, respectively. In a dropout layer, some of the weights may be randomly set to zero during training.

In some embodiments of the present disclosure, an advanced algorithm executed by processor 70 of self-tonometry device 30 may be used to eliminate the effect of eyelid 15 on the eye pressure measurements performed by the plurality of pressure sensors 20 in sensor array 50. Since the size of sensors 20 in sensor array 50 is small(2 mm×2 mm), the total area of sensor array 50 may be also small, such as, for example, 10 mm×10 mm. It may be assumed that all of pressure sensors 20 in array 50 may measure the same surface pressure of eye 10 except for minor variations introduced by the soft tissue of eyelid 15. Therefore, multiple sensors 20 in sensor array 50 may sample multiple respective pressures in eye 10 with the same underlying intraocular pressure, albeit at different respective positions 40 of eye 10 (e.g., on eyeball 15), which are determined by the position of sensors 20 in array 50 when placed onto the eye (e.g., outer surface 15A of eyelid 15) for IOP measurements.

If the measurement variation introduced by the eyelid is assumed to be random, multiple measurements made by sensors 20 in sensor array 50 may be used to cancel out the measurement variation for computing the IOP. Even if the eyelid variation is not random, as long as it has a consistent spatial relation to the sensor array (e.g., eyelid 15 may reduce the measured pressure more at the center and less on the edges of array 50). ANN 100 may automatically correct for this error by removing the pressure offset introduced by the eyelid. The ANN is a universal function approximator.

In some embodiments of the present disclosure, these dynamic properties may be used. For example, an accelerometer may be attached to self-tonometry device 30, and a force applied to the eyeball, such as a periodic compression and relaxation of the eyeball. The periodic force may be produced by finger 35 or by any suitable actuator or motor coupled to force-transfer assembly 25 either internally and/or externally to self-tonometry device 30. The accelerator on self-tonometry device 30 may be used to measure how fast eyeball 15 is being compressed. This information may be used with the geometry of pressure sensor array 50 to assess how eye 10 reacts to the applied force. Eyeball 15 with a higher IOP may be more resistant to compression; hence, the pressure values measured by sensor array 50 in self-tonometry device 30 may rise faster given the same compression velocity. The pressure values may return to the resting state faster during relaxation due to the higher IOP. Both the accelerator reading and pressure sensors reading may be input to the machine learning module such as ANN model 100 to compute the IOP.

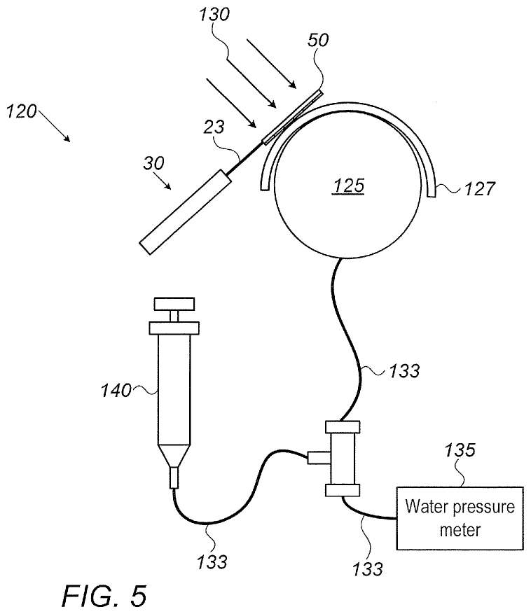

FIG. 5 schematically illustrates an experimental setup 120 for testing self-tonometry device 30 on a mechanical model, in accordance with some embodiments of the present disclosure. Experimental setup 120 may include a silicone ball 125 with a silicone sheet 127, a syringe 140 for controlling water pressure in silicone ball 125, tubing 133 for transporting the water, and a water pressure meter 135 for measuring the water pressure in silicone ball 125.

Silicone ball 125 may be used as an eye phantom for testing self-tonometry device 30. Silicone ball 125 may be filled with water and connected using tubing 133 to digital water pressure meter 135, which may record the intra-luminal pressure of the eye phantom in real-time. Syringe 140 filled with water may be coupled to experimental setup 120 to adjust the water pressure inside silicone ball 125. Thin silicone sheet 127 covered the eye phantom (e.g., silicone ball 125) to approximate the effect of eyelid 15 on the IOP measurements using self-tonometry device 30. Self-tonometry device 30 may be pressed against silicone ball 125 as shown by arrows 130 showing the direction of applied force of sensor array 50 on onto the model of the eye. The applied force may also be performed in a cyclic fashion repeatedly.

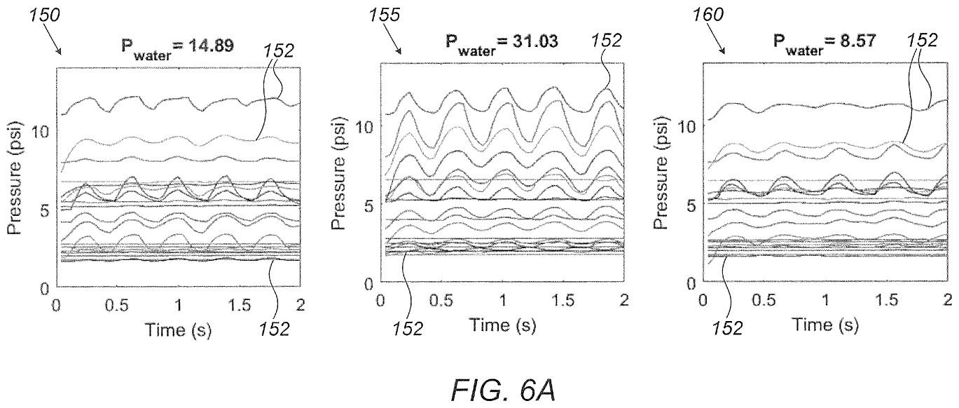

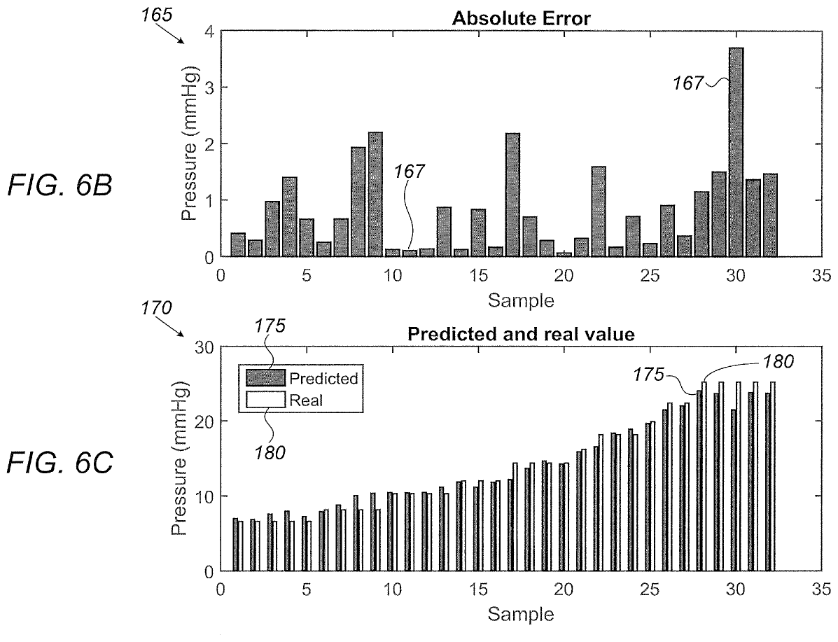

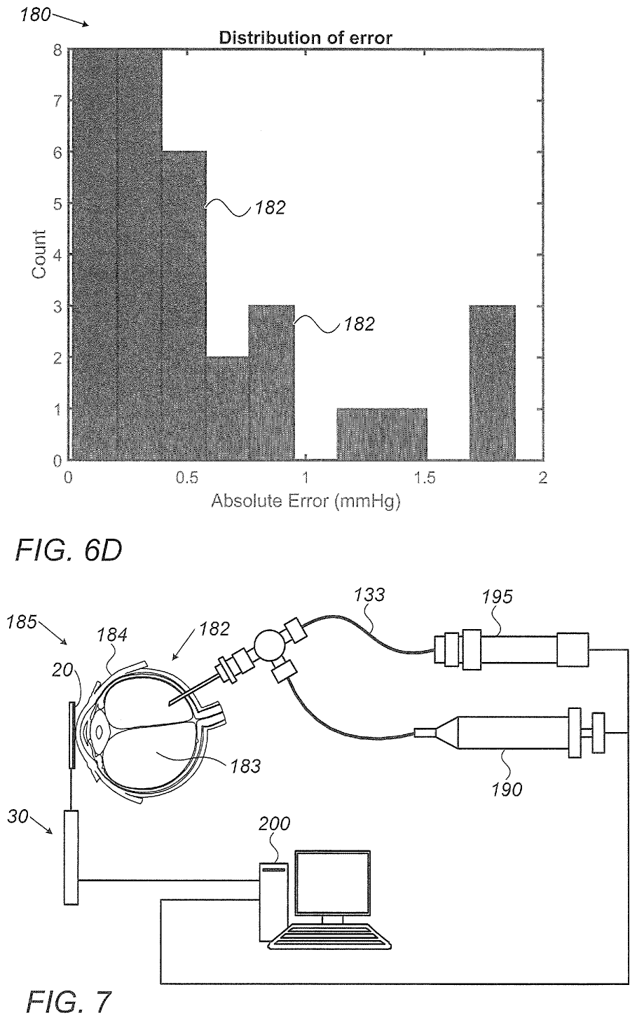

The water pressure in the eye phantom was recorded before the cyclic action was taken as the ground truth for training and testing MLM 71 in self-tonometry device 30. In experimental measurements shown in FIGS. 6A-6D below, 105 data samples may be taken where 70% of the samples trained MLM 71 in this case, a random forest model. The remaining data samples may be used for testing.

FIG. 6A illustrates graphs of sensor pressure measurements 152 versus time for different intra-luminal pressures on the mechanical model, according to some embodiments of the present disclosure. Different eye phantom intra-luminal pressures denoted P_{water} (e.g., the equivalent to IOP in a real eye) generated different patterns in sensor array readings 152, which may be used to infer real pressure from water pressure meter 135. A graph 150 shows the representative pressure traces for P_{water}=14.89 mmHg. A graph 155 shows the representative pressure traces for P_{water}=31.03 mmHg. A graph 160 shows the representative pressure traces for P_{water}=8.57 mmHg.

FIG. 6B illustrates a graph 165 of absolute error 167 versus data sample, in accordance with some embodiments of the present disclosure. Absolute error 167 is the error between the real and predicted intra-luminal pressure measured by self-tonometry device 30.

FIG. 6C illustrates a graph 170 of a real pressure 180 and a predicted pressure 175 versus data sample, in accordance with some embodiments of the present disclosure.

FIG. 6D illustrates a graph 180 of sample count 182 versus absolute error, in accordance with some embodiments of the present disclosure. The predicted pressure measured using self-tonometry device 30 closely follows the real pressure measured by a calibration device as shown in FIG. 6C. Most of the errors are below 1 mmHg as shown in FIG. 6D.

Furthermore, one feature of the self-measurement by a subject of IOP as taught herein is that the simultaneous intra-luminal pressure is not needed to train the computational model (e.g., MLM 71). The true pressure before sensor measurements may be taken as the ground truth for training the MLM 71, and the trained MLM 71 may be stored prior to use of self-tonometry device 30. Thus, using the embodiments taught herein, the ground truth of the IOP before taking sensor measurements may be obtained from more traditional IOP measurements such as using a Goldmann applanation tonometer.

FIG. 7 schematically illustrates an experimental setup 185 for testing self-tonometry device 30 on a pig eye 182, in accordance with some embodiments of the present disclosure. Experimental setup 185 may include a syringe 190 for injecting water into pig eyeball 183 to change the intra-luminal pressure, and a water pressure sensor 195 controlled by a computer 200. The computational model (e.g., MLM 71) for self-tonometry device 30 was validated using an animal subject, namely pig eye 182 with a pig eyeball 183 and pig eyelid 184. Experimental setup 185 is similar to the mechanical model shown in FIG. 5, except that a pig's eyeball was used to measure the IOP with self-tonometry device 30.

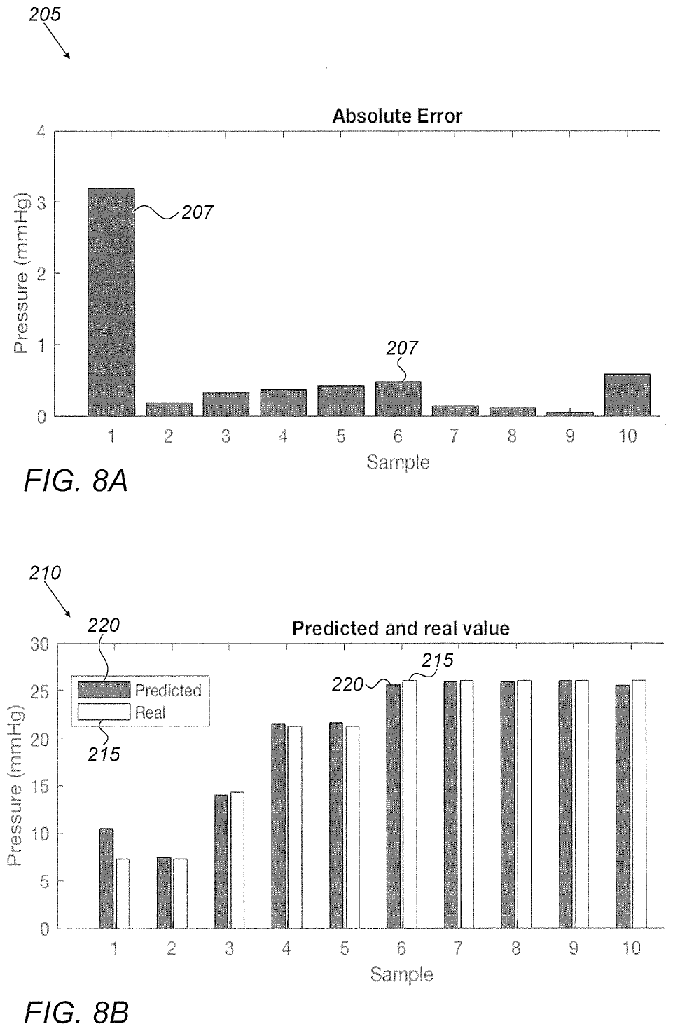

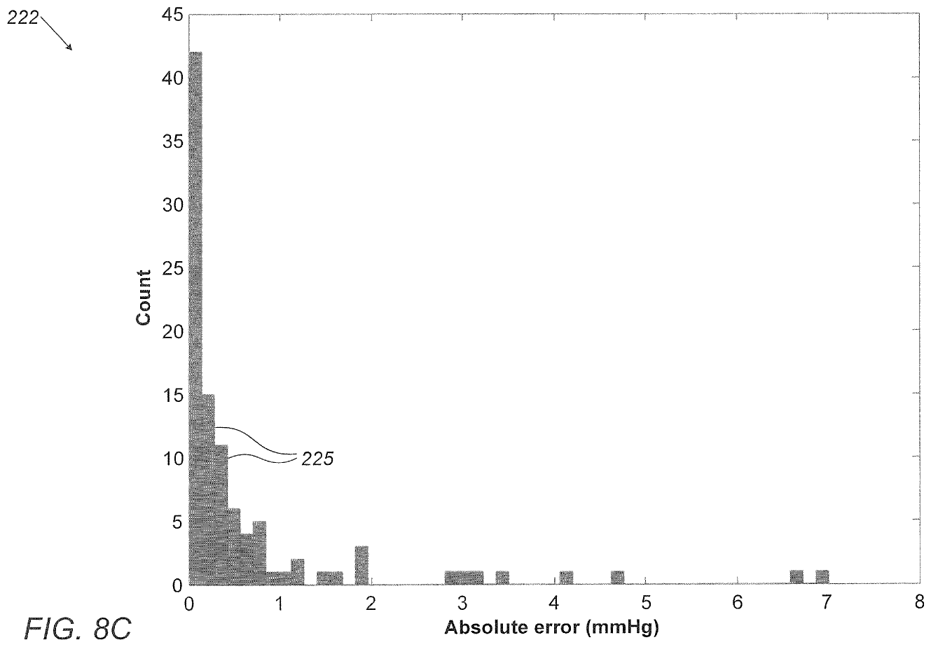

FIG. 8A illustrates a graph 205 of absolute error versus data sample, in accordance with some embodiments of the present disclosure. The absolute error is the error between the real and predicted intra-luminal pressure measured by self-tonometry device 30.

FIG. 8B illustrates a graph 210 of the real pressure and the predicted pressure versus data sample, in accordance with some embodiments of the present disclosure.

FIG. 8C illustrates a graph 222 of sample count 225 versus absolute error, in accordance with some embodiments of the present disclosure.

A predicted IOP 220 and a real IOP 215 in the animal model are shown in FIG. 8B. Most of the errors are within 2 mmHg as shown in FIG. 8C, which validated that self-tonometry device 30 reasonably estimated the IOP through sensors 20 placed on top of pig eyelid 184.

In some embodiments of the present disclosure, due to the individual difference in the eyelid thickness and mechanical property, self-tonometry device 30 may be calibrated for each patient for maximum accuracy. However, the device may estimate a reasonably accurate IOP reading without the need for individual calibration of self-tonometry device 30 for each subject.

For individually calibrating self-tonometry device 30, the subject's IOP may be measured using normal Goldmann applanation tonometry, for example, to obtain the standard IOP (e.g., real IOP). Pressure readings may be obtained by self-tonometry device 30 and ANN model 100 may then be calibrated using the standard IOP.

In some embodiments of the present disclosure, calibration data may be obtained in the APP/PC software for the device. The format of the calibration data may be as follows in Table I:

TABLE I
Representative Calibration Data

| Fiducial | | | | | | |
|---|---|---|---|---|---|---|
| Value | PSI(1) | ADC(1) | PSI(2) | ADC(2) | PSI(3) | ADC(3) |
| 18567 | 0 | 18484 | 0.952933 | 18619 | 1.91672 | 18802 |
| 18719 | 0 | 18638 | 0.952933 | 18789 | 1.91672 | 19021 |
| 19031 | 0 | 18988 | 0.952933 | 19188 | 1.91672 | 19490 |
| 19549 | 0 | 19483 | 0.952933 | 19657 | 1.91672 | 19932 |
| 19920 | 0 | 19878 | 0.952933 | 20017 | 1.91672 | 20226 |
| 20804 | 0 | 20733 | 0.952933 | 21005 | 1.91672 | 21285 |
| 18402 | 0 | 18572 | 0.952933 | 18900 | 1.91672 | 19255 |
| 18559 | 0 | 18586 | 0.952933 | 18853 | 1.91672 | 19086 |
| 18997 | 0 | 18988 | 0.952933 | 19393 | 1.91672 | 19690 |
| 19431 | 0 | 19420 | 0.952933 | 19956 | 1.91672 | 20298 |
| 20164 | 0 | 20119 | 0.952933 | 20630 | 1.91672 | 21001 |
| 21158 | 0 | 21091 | 0.952933 | 21726 | 1.91672 | 21955 |
| 22057 | 0 | 21963 | 0.952933 | 22384 | 1.91672 | 22603 |
| 20277 | 0 | 20207 | 0.952933 | 20742 | 1.91672 | 21046 |
| 19451 | 0 | 19436 | 0.952933 | 19868 | 1.91672 | 20206 |
| 19163 | 0 | 19117 | 0.952933 | 19453 | 1.91672 | 19715 |
| 18524 | 0 | 18530 | 0.952933 | 18996 | 1.91672 | 19248 |
| 18258 | 0 | 18312 | 0.952933 | 18900 | 1.91672 | 19215 |
| 20676 | 0 | 20645 | 0.952933 | 21093 | 1.91672 | 21430 |
| 19863 | 0 | 19830 | 0.952933 | 19981 | 1.91672 | 20229 |
| 19475 | 0 | 19428 | 0.952933 | 19563 | 1.91672 | 19774 |
| 18898 | 0 | 18865 | 0.952933 | 19022 | 1.91672 | 19279 |
| 18560 | 0 | 18521 | 0.952933 | 18709 | 1.91672 | 19030 |
| 18346 | 0 | 18290 | 0.952933 | 18462 | 1.91672 | 18710 |

In Table I, the pressure unit of PSI may be converted to mmHg. The output of ADC 95 implemented for example by analog IC chip may include a 16 bit resolution as shown in Table I.

In some embodiments of the present disclosure, when self-tonometry device 30 may be applied in the human eyes, sensors 20 may cover the range of pressure from 5-80 mmHg. Self-tonometry device 30 may measure the IOP by assessing the dynamic property of eye 10, similar to how repeatedly pressing a finger, for example, on an object may be used to assess its hardness. The absolute pressure applied on the eye is not the relevant parameter for determining IOP using MLM 71, only the change in the measured pressures in sensors in response to the applying a cyclic force with sensor array 50 contacting eye 10, which may be used by MLM 71 for estimating IOP. Stated differently, ANN model 100 may automatically infer the IOP by observing how the pressure of each sensor element 20 may change in response to periodic compression and relaxation.

As eyeball 15 is compressed, for example, the eyeball shape may respond differently under different internal IOP pressure. For the same applied force 130, such as a compression force for example, if the internal pressure of the eye is high, then eyeball 15 may not be easily deformed. The opposite is true for low pressure. Therefore, by processor 70 tracking the relation between applied pressure 130 and rate of deformation, for example, the internal pressure IOP of eye 10 may be determined to explain this phenomenon in simplified terms. However, in actuality, the relation between the two parameters of pressure and the deformation is highly complex and non-linear. Thus, MLM 71, such as ANN 100 model or a random forest model, may be used to infer the relation between these two parameters.

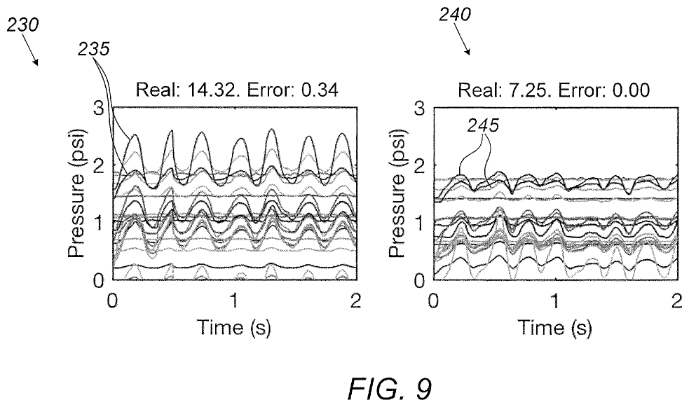

FIG. 9 illustrates graphs of sensor pressure measurements versus time using self-tonometry device 30 on a subject's eye 10, according to some embodiments of the present disclosure. A graph 230 shows representative pressure traces 235 from sensors 20 in sensor array 50. Processor 70 executing MLM 71 (e.g., ANN 100, for example) may use representative pressure traces 235 as input data to ANN 100 so as to estimate IOP in eye 10 as 14.32 mmHg with an estimation error of 0.34. A graph 240 shows representative pressure traces 245 from sensors 20 in sensor array 50. Processor 70 executing MLM 71 (e.g., ANN 100, for example) may use representative pressure traces 245 as input data to ANN 100 so as to estimate IOP in eye 10 as 7.25 mmHg with zero estimation error in this case.

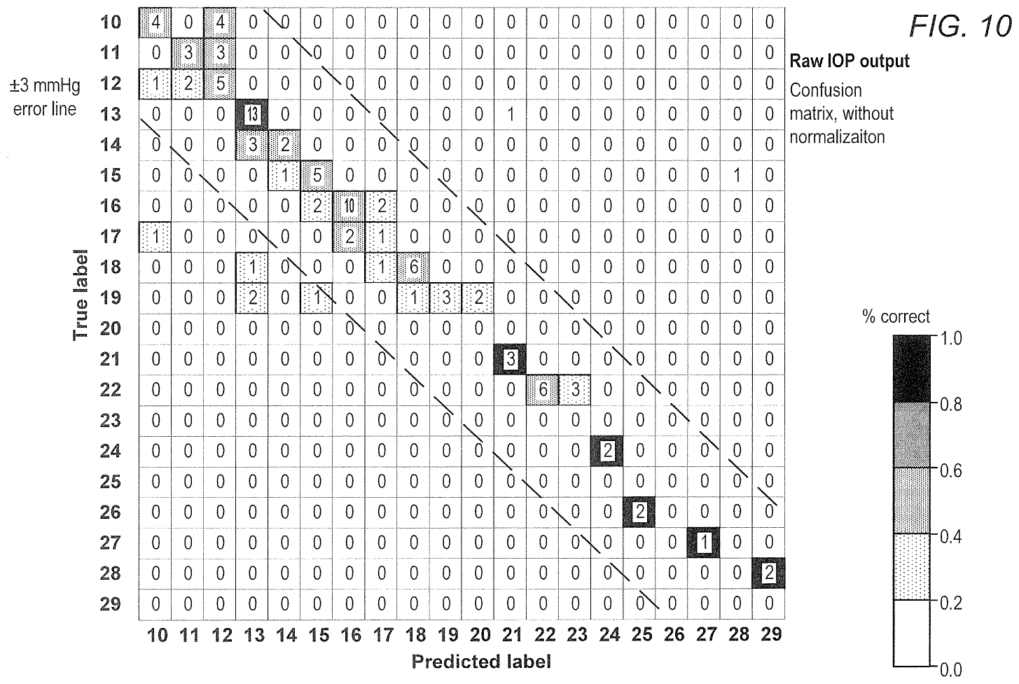

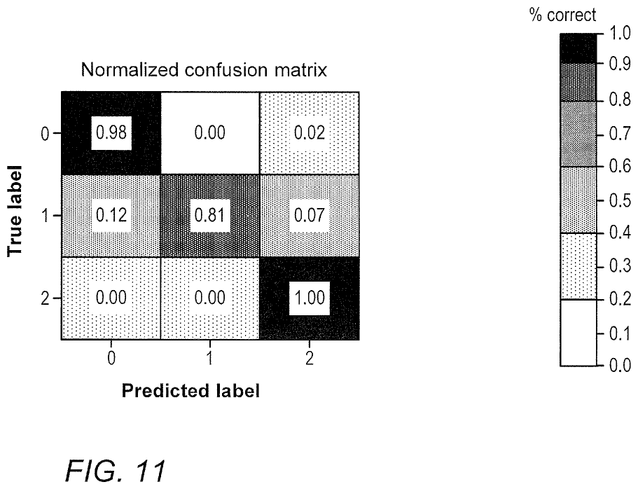

FIG. 10 illustrates a graph of sensor pressure measurements comparing predicted pressure with true actual tested pressure using self-tonometry device 30 on a subject's eye 10, according to some embodiments of the present disclosure. According to some embodiments, self-tonometry device 30 may be trained according to reference data. e.g., by ANN model 100, followed by a test of their IOP in eye 10 using self-tonometry device 30. In some embodiments, the first two sessions are training sessions based on the reference data. The last session is the testing of self-tonometry device 30. The results are within an error of ±3 mmHg and the trials where the model was uncertain, for example, that their probability was below 0.7, were not included in the graph of FIG. 10. The graph does not include normalization, unlike the graph in FIG. 11.

FIG. 11 illustrates a graph of normalized sensor pressure measurements comparing predicted pressure with true actual tested pressure using self-tonometry device 30 on a subject's eye 10, according to some embodiments of the present disclosure. Pressure results below 15 mmHg were attributed with zero, pressure results between 15 to 20 mmHg were attributed with one, and pressure results above 20 mmHg were attributed with two. The percentage of correctness is also indicated by the color of each square of the graph, as well as being indicated inside each square, from 0 to 1.0. It is clear that measurements of pressure above 20 mmHg, meaning measurements attributed with two in both the predicted value and the true value, are highly accurate, indicating a correctness percentage of 1.0. Measurements of pressure below 15 mmHg are also highly accurate with a correctness percentage of 0.98 in measurements attributed with zero in both the predicted label and the true label, as are measurements of pressure between 15-20 mmHg attributed with one in both the predicted label and the true label, which have a correctness percentage of 0.81. However, it is clear that the correctness percentage is zero or slightly above zero, in cases where the predicted value is different from the real value, e.g., when the predicted value is one, while the true value is 2. The normalized graph of FIG. 11 makes it easier to notice that there is high concordance between the predicted value of pressure and that tested by self-tonometry device 30.

In some embodiments of the present disclosure, a self-tonometry device for measuring intra-ocular pressure in an eye of a subject may include a plurality of sensors and a processor for executing a machine learning module. The plurality of sensors may be arranged in an array for measuring a plurality of pressures at respective positions on an eye of a subject, when the plurality of sensors in the array apply a force to the eye through an eyelid of the subject. The processor may be configured to receive the plurality of pressures at the respective positions measured from the plurality of sensors as an input to the machine learning module, and to compute using the machine learning module, an intra-ocular pressure in the eye based on the plurality of pressures at the respective positions measured through the eyelid of the subject.

1. A self-tonometry device for measuring intra-ocular pressure (IOP) in an eye of a subject, the self-tonometry device comprising:
a plurality of sensors arranged in an array for measuring a plurality of pressures at respective positions on the eye of the subject, when the plurality of sensors in the array apply a force to the eye through an eyelid of the subject; and
a processor for executing a machine learning module, which is configured to receive the plurality of pressures at the respective positions measured from the plurality of sensors as an input to the machine learning module, and to compute using the machine learning module, the intra-ocular pressure in the eye based on the plurality of pressures at the respective positions measured through the eyelid of the subject.

2. The self-tonometry device according to claim 1, wherein the machine learning module comprises an artificial neural network model or a random forest model.

3. The self-tonometry device according to claim 2, wherein the artificial neural network is trained and calibrated by comparing the plurality of pressures measured by the plurality of sensors to true IOP measured using a reference device.

4. The self-tonometry device according to claim 2, wherein the artificial neural network is calibrated using gradient descent.

5. The self-tonometry device according to claim 2, wherein the artificial neural network is calibrated per subject.

6. The self-tonometry device according to claim 2, wherein the processor computes the intra-ocular pressure in the eye by:
(a) feeding the measured pressures into the artificial neural network;
(b) transforming the measured pressures by one or multiple convolutional layers to produce an output;
(c) transforming the output by a normalization layer;
(d) transforming the output by one or multiple convolution layers;
(e) transforming the output by a normalization layer;
(f) transforming the output by a dropout layer;
(g) transforming the output by one or multiple fully connected layer; and
(h) estimating the intra-ocular pressure in the eye.

7. The self-tonometry device according to claim 1, wherein the intra-ocular pressure in an eye of a subject is a nonlinear function of the pressures measured by the plurality of sensors, given by Eqn. (2):
$${p}_{I} = {v}^{T} {a}^{( 1 )} + {b}^{( 2 )} = {v}^{T} \frac{1}{1 + exp ( - {ω}^{T} X + {b}^{( 1 )} ) )} + {b}^{( 2 )} = {\sum }_{m = 1}^{M} ( \frac{{v}_{m}}{1 + exp ( - ( {\sum }_{i = 1}^{24} {ω}_{mi} {p}_{i} + {b}_{m}^{( 1 )} ) )} + {b}^{( 2 )} ) \qquad ( 2 )$$
whereby
Pᵢ is the pressure measured by each of the plurality of sensors 1 to 24;
M is the number of hidden neurons optimized using experimental data;
ω=(ω1,1, ω1,2, . . . , ω24,M)T is the matrix of the weights connecting the nodes in input layer with neurons of hidden layer;
b(1)=(b1(1), b2(1), . . . , bM(1))T is the bias vector of the neurons of hidden layer;
V=(v1, v2, . . . , vM)T is the vector of the weights connecting the neurons of hidden layer with those in output layer; and
b(2) is the bias of the neuron of output layer.

8. The self-tonometry device according to claim 1, further comprising a force transfer assembly configured to applying the force by a finger of the subject.

9. The self-tonometry device according to claim 1, wherein the plurality of sensors comprise capacitive pressure sensors.

10. The self-tonometry device according to claim 1, wherein the plurality of sensors are coupled to a flexible member at an end of the self-tonometry device.

11. The self-tonometry device according to claim 10, wherein said flexible member permits the plurality of sensors to be pushed into contact with the eyelid.

12. The self-tonometry device according to claim 1, further comprising a display for displaying the intra-ocular pressure or other metrics.

13. The self-tonometry device according to claim 1, wherein said plurality of pressures at respective positions on the eye of the subject are multiple pressure readings from different positions used to improve the accuracy of the intra-ocular pressure measurement.

14. The self-tonometry device according to claim 1, wherein the thickness of the array of said plurality of sensors is 0.5 mm such to be flexible to press.

15. The self-tonometry device according to claim 1, further comprising an accelerator for measuring how fast an eyeball of the subject is being compressed.

16. A method for measuring intra-ocular pressure in an eye of a subject, the method comprising:
measuring a plurality of pressures at respective positions on the eye of the subject using a plurality of sensors arranged in an array, when the plurality of sensors in the array apply a force to the eye through an eyelid of the subject;
in a processor executing a machine learning module, receiving the plurality of pressures at the respective positions measured from the plurality of sensors as an input to the machine learning module; and
computing in the processor using the machine learning module, the intra-ocular pressure in the eye based on the plurality of pressures measured through the eyelid of the subject.

17. The method according to claim 16, wherein the machine learning module comprises an artificial neural network model or a random forest model.

18. The method according to claim 17, wherein the method further comprises training the artificial neural network by comparing the plurality of pressures measured by the plurality of sensors to true IOP measured using a reference device.

19. The method according to claim 17, wherein the method further comprises calibrating the artificial neural network using gradient descent.

20. The method according to claim 19, wherein the calibrating is per subject.

21. The method according to claim 17, wherein the computing in the processor using the machine learning module comprises:
(a) feeding the measured pressures into the artificial neural network;
(b) transforming the measured pressures by one or multiple convolutional layers to produce an output;
(c) transforming the output by a normalization layer;
(d) transforming the output by one or multiple convolution layers;
(e) transforming the output by a normalization layer;
(f) transforming the output by a dropout layer;
(g) transforming the output by one or multiple fully connected layer; and
(h) estimating the intra-ocular pressure in the eye.

22. The method according to claim 16, further comprising applying the force by a finger of the subject with a force transfer assembly.

23. The method according to claim 16, wherein the plurality of sensors comprise capacitive pressure sensors.

24. The method according to claim 16, wherein the intra-ocular pressure in an eye of a subject is a nonlinear function of the pressures measured by the plurality of sensors, given by Eqn. (2):
$${p}_{I} = {v}^{T} {a}^{( 1 )} + {b}^{( 2 )} = {v}^{T} \frac{1}{1 + exp ( - {ω}^{T} X + {b}^{( 1 )} ) )} + {b}^{( 2 )} = {\sum }_{m = 1}^{M} ( \frac{{v}_{m}}{1 + exp ( - ( {\sum }_{i = 1}^{24} {ω}_{mi} {p}_{i} + {b}_{m}^{( 1 )} ) )} + {b}^{( 2 )} ) \qquad ( 2 )$$
whereby
Pᵢ is the pressure measured by each of the plurality of sensors 1 to 24;
M is the number of hidden neurons optimized using experimental data;
ω=(ω1,1 ω1,2, . . . , ω24,M)T is the matrix of the weights connecting the nodes in input layer with neurons of hidden layer;
b(1)=(b1(1), b2(1), . . . , bM(1))T is the bias vector of the neurons of hidden layer;
V=(v1, v2, . . . , vM)T is the vector of the weights connecting the neurons of hidden layer with those in output layer; and
b(2) is the bias of the neuron of output layer.
